(19) United States
(12) Patent Application Publication
(10) Pub. No.: US 2007/0288036 A1
(43) Pub. Date: Dec. 13, 2007

(54) ASSEMBLY FOR CROSSING A CHRONIC TOTAL OCCLUSION AND METHOD THEREFOR

(76) Inventors: Niranjan Seshadri, Santa Fe, NM (US); Bryan Brazil, Las Vegas, NM (US); Nagraj Seshadri, Ridgewood, NJ (US)

Correspondence Address: WEISS & MOY PC, 4204 NORTH BROWN AVENUE, SCOTTSDALE, AZ, 85251, US

(21) Appl. No.: 11/423,402

(22) Filed: Jun. 9, 2006

(51) Int. Cl. A61B 17/22

(52) U.S. Cl. 606/127; 606/159

(57) ABSTRACT

An assembly and method for treatment for crossing hardened plaque, such as that present in chronic total occlusions. The assembly comprises a centering catheter and a crosser dimensioned to pass through the centering catheter. The crosser has a substantially pointed tip to engage and penetrate the hardened plaque. And the crosser is dimensioned such that twisting the crosser will help to advance the crosser through the plaque.

### FIELD OF THE INVENTION

This invention relates generally to devices and procedures for medical care and pertains in particular to medical devices and techniques for treatment of chronic total occlusions.

### BACKGROUND OF THE INVENTION

A total occlusion occurs when there is no blood flow through an artery because of blockage from hardened, rock-like plaque. If the blockage has been present for approximately a week, the total occlusion is termed to be “acute.” However, a chronic total occlusion is one that has been present for approximately three or more months or even years. Such chronic total occlusions may develop within the heart or within peripheral arteries in the arms and legs.

Because the chronic total occlusion is comprised of such hardened plaque, it is difficult for a guidewire to pass through it. Therefore, in order to treat it, the chronic total occlusion must first be crossed. In other words, a passage must be cut through the chronic total occlusion. Once the chronic total occlusion is crossed, a physician will follow up by performing an angioplasty, implanting a stent, or performing plaque excision.

There currently exist some chronic total occlusion crossers, such as the LUMEND FRONTRUNNER, that use a reverse scissor spreading-type action, also referred to as blunt micro-dissection, to separate the plaque. A major problem with this type of device is that there is no centering aspect to ensure that the device remains centered within the lumen of the artery. Without a centering device, there is great danger of perforation of the artery wall. In addition, it may be difficult to enter the lesion due to the blunt nature of the tip of the device.

Other devices, such as the ASAHI TORNUS catheter, have several hair-thin, stainless steel strands braided together to enhance flexibility and strength. A common problem with this type of device, however, is that it has a dull tip that is inadequate to penetrate the hardened rock-like plaque of a chronic total occlusion, and it requires a wire to cross initially before it can be advanced over the wire.

Therefore a need existed for an assembly and method for crossing a chronic total occlusion. Preferably the assembly will be dimensioned to remain centered within the lumen of the artery during crossing. Further preferably, the assembly would reduce the likelihood of perforation of the artery wall during crossing. Still further preferably, the assembly will be dimensioned to more effectively penetrate a chronic total occlusion.

### SUMMARY OF THE INVENTION

An object of the present invention is to provide an assembly and method for crossing a chronic total occlusion.

It is another object of the present invention to provide an assembly and method therefor for crossing a chronic total occlusion that is dimensioned to remain centered within the lumen of the artery during crossing.

It is another object of the present invention to provide an assembly and method for crossing a chronic total occlusion that will reduce the likelihood of perforation of the artery wall during crossing.

It is still another object of the present invention to provide an assembly and method that is more effective in penetrating a chronic total occlusion.

### BRIEF DISCRIPTION OF THE PREFERRED EMBODIMENTS

In accordance with one embodiment of the present invention, an assembly for crossing hardened plaque is disclosed. The assembly comprises a centering catheter and a crosser. The crosser has means for crossing the hardened plaque and is dimensioned to pass through the centering catheter.

In accordance with another embodiment of the present invention, an assembly for crossing a chronic total occlusion is disclosed. The assembly comprises a centering catheter and a crosser. The crosser is dimensioned to be passed through the centering catheter and the crosser comprises a proximal end, a shaft portion coupled to and extending from the proximal end of the crosser, a hollow lumen defined by and extending along the length of the crosser, and a corkscrew-shaped distal end coupled to and extending from the shaft portion of the crosser.

In accordance with another embodiment of the present invention, a method for crossing a chronic total occlusion is disclosed. The method comprises the step of providing an assembly having a centering catheter having a centering balloon coupled about a shaft portion of the centering catheter and coupled proximate a distal end of the centering catheter and having a crosser having means for crossing the chronic total occlusion, the crosser being dimensioned to pass through the centering catheter. The method further comprises the steps of advancing a guide wire through an artery to a site proximate the chronic total occlusion, advancing the centering catheter over the guide wire and through the artery to the site proximate the chronic total occlusion, inserting the crosser into the centering catheter over the guide wire, inflating the centering balloon of the centering catheter within the artery, advancing the means for crossing the chronic total occlusion through an aperture defined by the distal end of the centering catheter, and engaging the chronic total occlusion with the means for crossing the chronic total occlusion.

In accordance with yet another embodiment of the present invention, a method for crossing a chronic total occlusion is disclosed. The method comprises the step of providing an assembly having a centering catheter and a crosser dimensioned to be passed through the centering catheter and comprising a proximal end, a shaft portion coupled to and extending from the proximal end of the crosser, a hollow lumen defined by and extending along the length of the crosser, a handle coupled to the shaft portion of the crosser, and a corkscrew-shaped distal end coupled to and extending from the shaft portion of the crosser. The method comprises the additional steps of advancing a guide wire through an artery to a site proximate the chronic total occlusion, advancing the centering catheter over the guide wire and through the artery to the site proximate the chronic total occlusion, inserting the crosser into the centering catheter over the guide wire, inflating a centering balloon of the centering catheter within the artery, twisting the handle to advance the corkscrew-shaped distal end of the crosser through an aperture defined by a distal end of the centering catheter, and engaging the chronic total occlusion with the corkscrew-shaped distal end of the crosser.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

FIG. 1 is a perspective view of an assembly for crossing a chronic total occlusion, in accordance with the present invention.

FIG. 1A is a perspective view of the assembly of FIG. 1, the only difference is that the interior of the centering catheter and the interior of the crosser are shown in phantom lines.

FIG. 2 is a perspective view of a centering catheter of the assembly of FIG. 1. Only the centering catheter is shown and its interior is shown in phantom lines.

FIG. 3 is a perspective view of a crosser of the assembly of FIG. 1. The only difference from the crosser shown in FIG. 1 is that the distal end of the crosser of this figure is shown as having helicoidal threads.

FIG. 4 is a perspective view of the distal end of the crosser of the assembly of FIG. 1. The interior of the shaft portion of the crosser is shown in phantom lines.

FIG. 5 is a perspective view of the assembly of FIG. 1. The distal end of the centering catheter, however, is not shown.

FIG. 6 is a perspective exploded view of the proximal end of the crosser of the assembly of FIG. 1. The only difference is that a guide wire is not shown passing through the crosser.

FIG. 6A is a perspective view of the proximal end of the crosser of the assembly of FIG. 1.

FIG. 7 is a cross-sectional view of the assembly of FIG. 1. The assembly is shown inserted into an artery.

FIG. 8 is a perspective view of the distal end of the crosser of the assembly of FIG. 1. This figure is similar to that shown in FIG. 4, the only difference being that the interior of the shaft portion of the crosser is not shown.

FIG. 9 is a perspective view of a second embodiment of the distal end of the crosser of the assembly of FIG. 1. The difference is that the distal end is shown as having helicoidal threads.

FIG. 10 is a perspective view of a third embodiment of the distal end of the crosser of the assembly of FIG. 1. The difference is that the distal end is shown as being auger-shaped.

FIG. 11 is a perspective view of a fourth embodiment of the distal end of the crosser of the assembly of FIG. 1. The difference is that the distal end is shown as having helicoidal threads and a tapered tip.

FIG. 12 is a perspective view of a fifth embodiment of the distal end of the crosser of the assembly of FIG. 1. The difference is that the distal end is shown as being substantially concave and substantially oblong.

FIG. 13 is a perspective view of a sixth embodiment of the distal end of the crosser of the assembly of FIG. 1. The difference is that the distal end is shown as being substantially concave and substantially oblong. The distal end is also shown as having a tapered tip.

FIG. 14 is left perspective view of a hemostatic valve coupled to the proximal end of the catheter of the assembly of FIG. 1. A portion of the crosser is shown passing therethrough.

FIG. 15 is a right perspective view of the hemostatic valve and crosser of FIG. 14.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

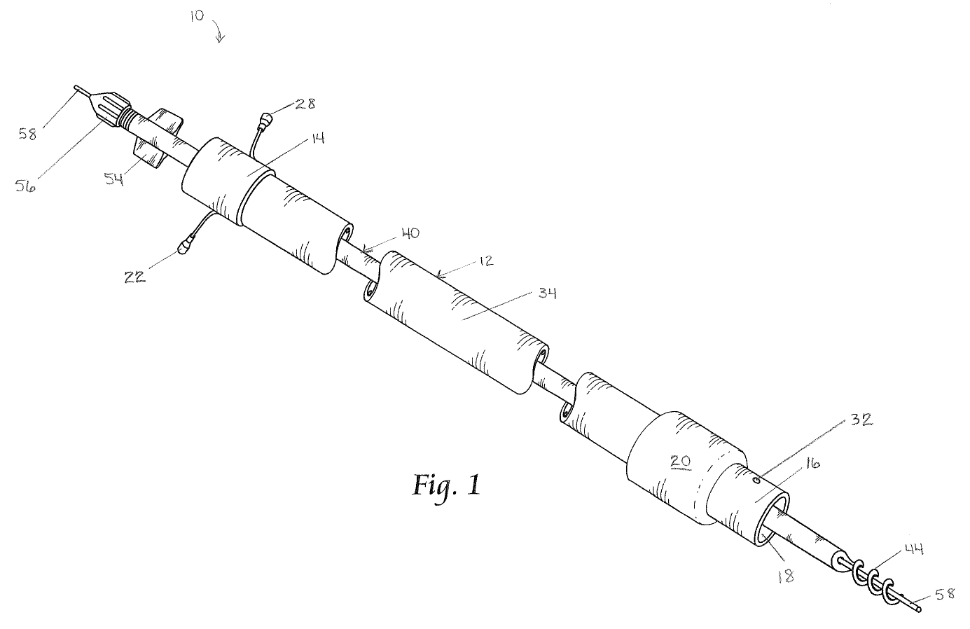

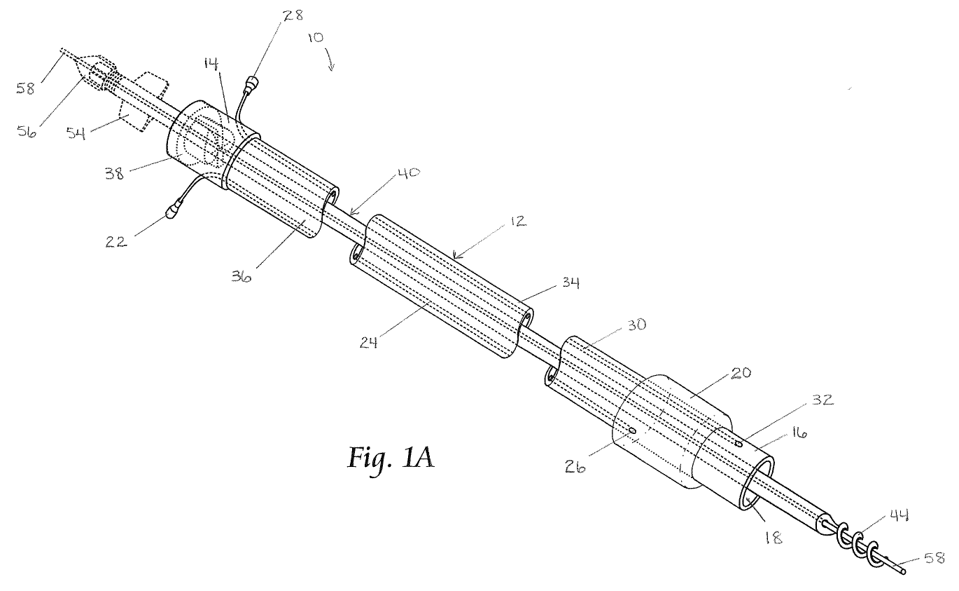

FIGS. 1-15 together disclose an assembly, referred to hereinafter as assembly 10, for crossing hardened plaque, such as that present in chronic total occlusions. The assembly 10 preferably comprises a centering catheter 12 and a crosser 40.

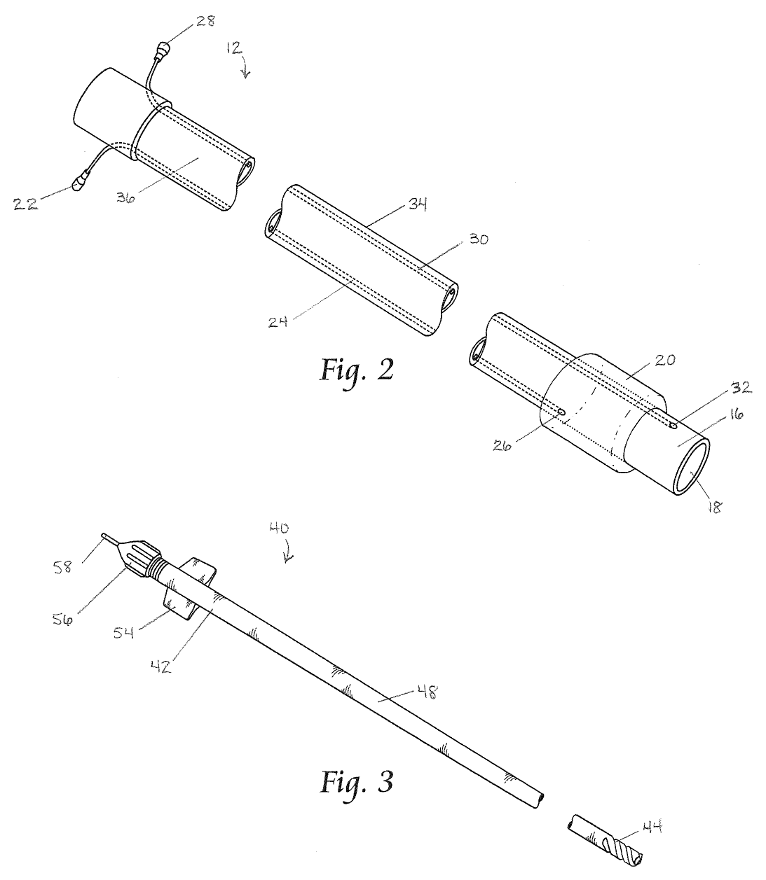

Referring to FIGS. 1-2, the centering catheter 12 has a proximal end 14, a shaft portion 34, and a distal end 16. Preferably, a centering balloon 20 is coupled about the shaft portion 34 of the centering balloon 20, proximate its distal end 16. While it is preferred that the centering catheter 12 use a centering balloon 20 coupled proximate its distal end 16, it should be clearly understood that substantial benefit may be derived from the use of alternative centering means and/or alternative placement of the centering means on the centering catheter 12, so long as it allows the centering catheter 12 to remain centered within the artery 60 (see FIG. 7).

In order to inflate the centering balloon 20, saline and radiographic contrast, or radiographic contrast alone, is inserted into a balloon port 22, through a lumen 24 (shown in FIGS. 1A and 2; see also FIG. 7) running along the length of the shaft 34 of the centering catheter 12, and through an aperture 26 (shown in FIGS. 1A and 2) that opens into the centering balloon 20. While it is preferred that the lumen 24 be integral to the interior of the shaft 34 of the centering catheter 12, it should be clearly understood that substantial benefit may be derived from a lumen 24 that is separate from the interior of the shaft 34 of the centering catheter 12. To deflate the centering balloon 20, the saline and radiographic contrast, or the radiographic contrast alone, travels in the opposite direction and is released from the balloon port 22.

During surgery, blood and debris will most likely build up in the site of the chronic total occlusion. In order to remove the blood and debris from the site of the chronic total occlusion, the blood and debris are aspirated. The blood and debris are withdrawn from the site through an aperture 32 preferably defined by the distal end 16 of the centering catheter 12. The blood and debris then pass through a lumen 30 (shown in FIGS. 1A and 2; see also FIG. 7) running along the length of the shaft 34 of the centering catheter 12 and finally exit through an aspiration port 28 preferably coupled to the proximal end 14 of the centering catheter 12. While it is preferred that the lumen 30 be integral to the interior of the shaft 34 of the centering catheter 12, it should be clearly understood that substantial benefit may be derived from a lumen 30 that is separate from the interior of the shaft 34 of the centering catheter 12.

Preferably, in an effort to control the amount of blood loss of the patient, a hemostatic valve 38 (shown in FIGS. 1A, 14 and 15) will be coupled to the proximal end 14 of the centering catheter 12. It should be clearly understood, however, substantial benefit may be derived from alternative forms of one-way valves, so long as the valve helps to prevent excess blood loss. It should also be understood that while the use of such a one-way valve is preferred, it is not required.

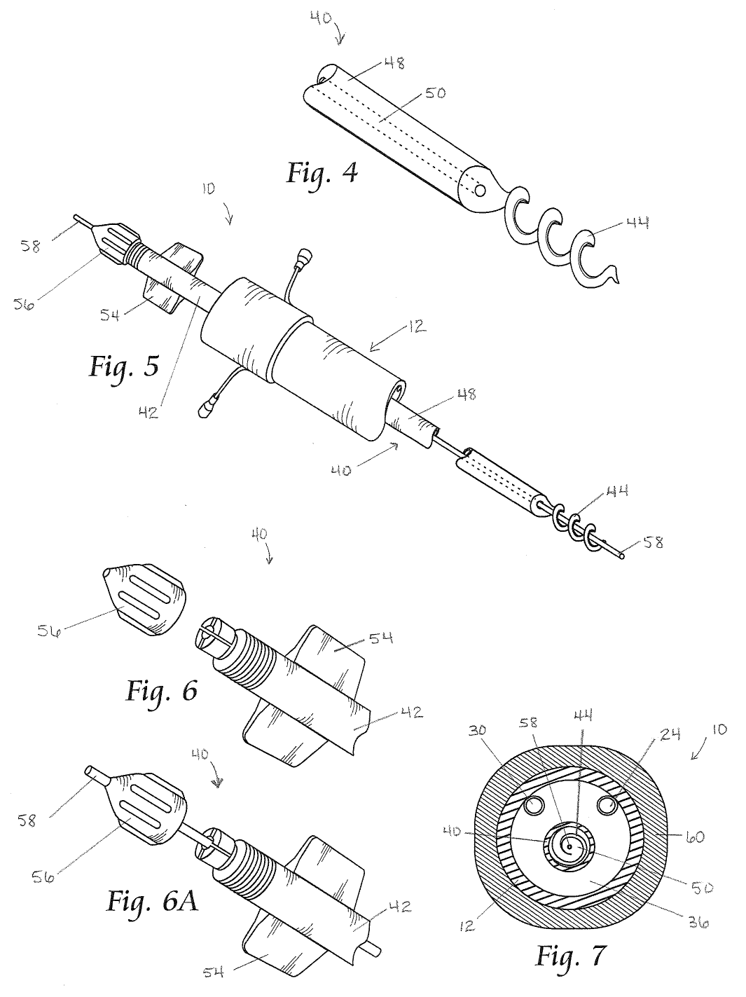

Referring now to FIGS. 3-7, the crosser 40 is dimensioned to pass through the lumen 36 (shown in FIG. 7; see also FIGS. 1A and 2) of the centering catheter 12. The crosser 40 preferably has a proximal end 42 (shown in FIGS. 3, 5, 6 and 6A), a shaft portion 48 (shown in FIGS. 3-5), a lumen 50 (shown in FIGS. 4 and 7) extending along the length of the crosser 40, and a distal end 44 (shown in FIGS. 3-5 and FIG. 7; see also FIGS. 1 and 1A). The lumen 50 is dimensioned to allow a guide wire 58 (shown in FIGS. 3, 5, 6A, and 7; see also FIGS. 1 and 1A) to pass therethrough.

It is preferable that the crosser 40 have a handle 54 (shown in FIGS. 3, 5, 6, and 6A; see also FIGS. 1 and 1A) that may either be removably coupled to or integral to its proximal end 42. The handle 54 aids the user in maneuvering and twisting the crosser 40 as it is advanced through the hardened plaque of the chronic total occlusion. It should be clearly understood, however, that substantial benefit may still be derived from a crosser 40 that does not have a handle 54 and from a crosser 40 that has a handle 54 coupled to a part of the crosser 40 other than its proximal end 42.

Still further preferably, the crosser 40 has tightening means, such as a torque device 56 (shown in FIGS. 3, 5, 6 and 6A; see also FIGS. 1 and 1A), that may be coupled to or integral to its proximal end 42. The torque device 56 will be used to secure the guide wire 58 in place when it is inserted through the crosser 40. Although it should be clearly understood that substantial benefit may still be derived from a crosser 40 that does not have a torque device 56 and from a crosser 40 that uses alternative tightening means.

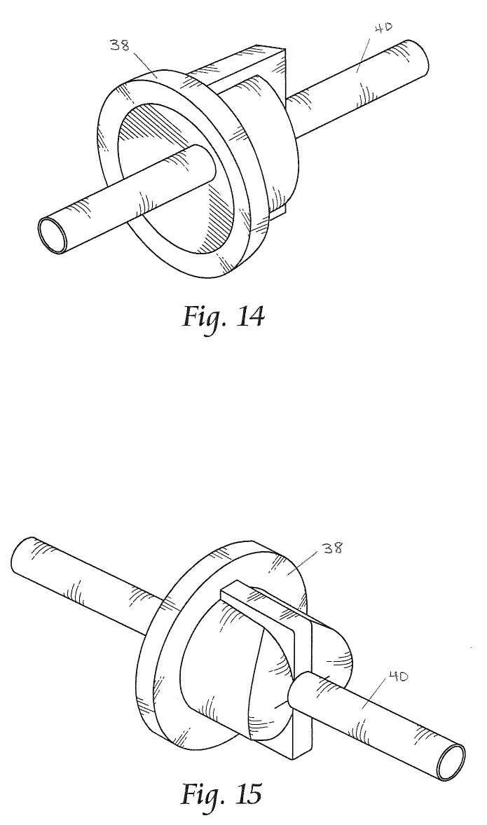

Referring to FIGS. 8-13, the crosser 40 of the assembly 10 of the present invention has means for crossing the hardened plaque of the chronic total occlusion. In order to aid crossing, it is preferable that the distal end 44 have at least one substantially pointed tip 52. Substantial benefit may be derived from the distal end 44 having one pointed tip 52 (shown in FIGS. 8-11) or more than one pointed tip 52 (shown in FIGS. 12-13). Having such a pointed tip 52 allows for more effective penetration and crossing of the hardened plaque.

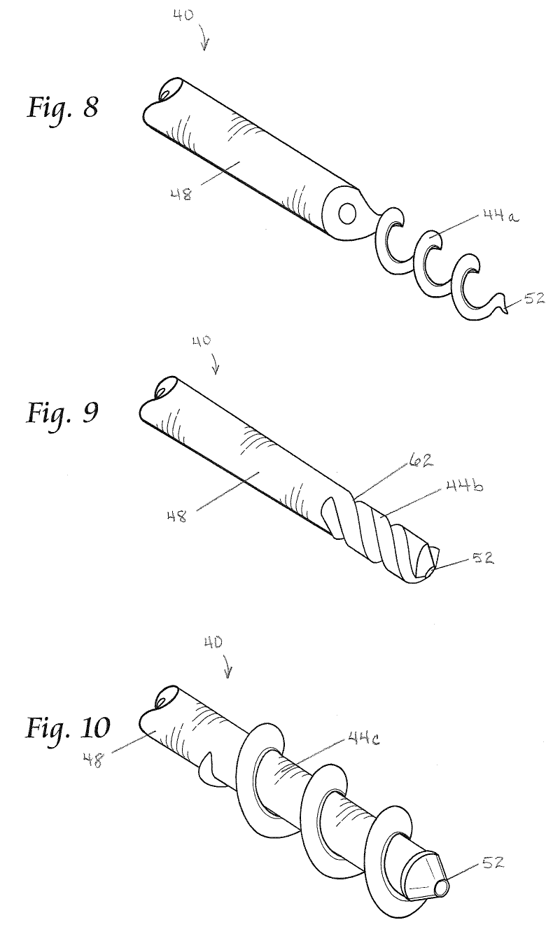

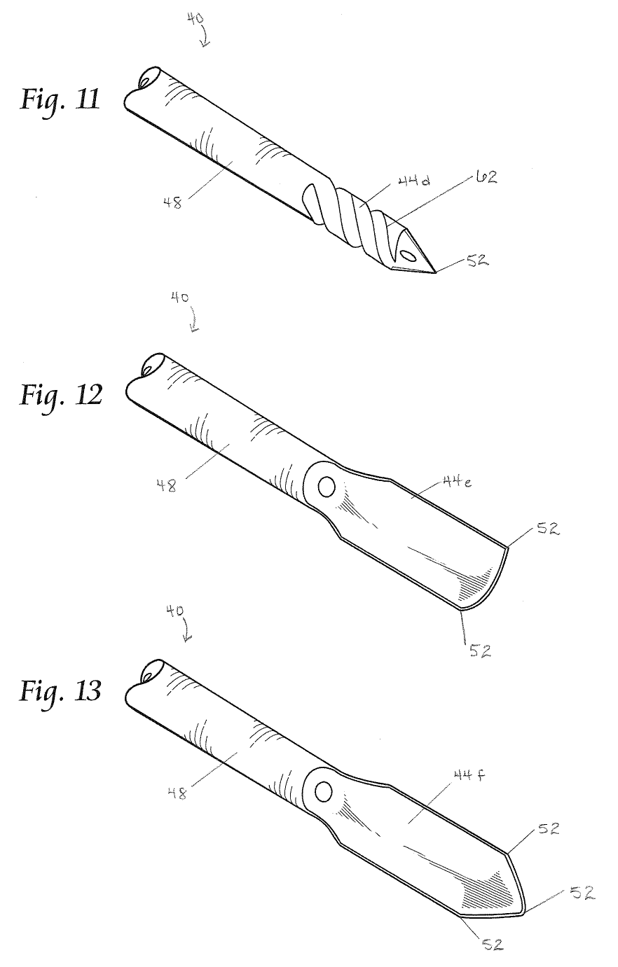

As shown in FIGS. 8-11, the distal end 44 of the crosser 40 may be dimensioned in a substantially spiral shape. For example, FIG. 8 shows a corkscrew-shaped distal end 44a (referred to generically as distal end 44), FIG. 9 shows a distal end 44b (referred to generically as distal end 44) having helicoidal threads 62, and FIG. 10 shows an auger-shaped distal end 44c (referred to generically as distal end 44). FIG. 11 shows a distal end 44d (referred to generically as distal end 44) having helicoidal threads 62 and a substantially tapered tip. Or, as shown in FIGS. 12-13, the distal end 44 of the crosser 40 may be substantially oblong and concave. FIG. 12 shows a distal end 44e (referred to generically as distal end 44) that is substantially oblong and concave and having two pointed tips 52 to engage the hardened plaque. And FIG. 13 shows a distal end 44f (referred to generically as distal end 44) being substantially oblong and concave and having a substantially tapered tip. It should be clearly understood that substantial benefit may be derived from the distal end 44 of the crosser 40 having an alternative shape so long as it has at least one pointed tip 52 to help penetrate the hardened plaque.

### Statement of Operation

In order to use the assembly 10 of the present invention, a guide wire 58 is preferably advanced through the artery 60 to a site proximate the chronic total occlusion. The centering catheter 12 is advanced over the guide wire 58 and through the artery 60 to the site proximate the chronic total occlusion. Once the centering catheter 12 is in place, the crosser 40 is inserted into the centering catheter 12 over the guide wire 58 and the centering balloon 20 is inflated within the artery 60. The distal end 44 of the crosser 40 is then advanced out of an aperture 18 (see FIGS. 1-2) defined by the distal end 16 of the centering catheter 12 and advanced toward the site of the chronic total occlusion. By twisting the crosser 40, its distal end 44 is better able to engage and penetrate the hardened plaque. The handle 54 would allow for better grip on the crosser 40 while twisting it. The guide wire 58 will still be in place and the chronic total occlusion may also be crossed with the guide wire 58 aided by the crosser 40.

As the crosser 40 advances toward the site of the chronic total occlusion and eventually through the plaque, another guide wire 58 may be passed through the lumen 50 and out the distal end 44 of the crosser 40. The guide wire 58 would therefore also be advanced toward the site of the chronic total occlusion and would eventually be advanced through the plaque. It should be clearly understood that advancement of the guide wire 58 may be performed simultaneously with or subsequent to the advancement of the crosser 40 through the plaque. It should also be clearly understood that, unlike other crossers, a guide wire 58 is not needed in order to advance the crosser 40 of the present invention through the plaque.

Once the guide wire 58 has passed through the chronic total occlusion, the centering balloon 20 may be deflated and the crosser 40 may be withdrawn from the site of the chronic total occlusion. Finally, an angioplasty, plaque excision, or a stent may be performed.

While the invention has been particularly shown and described with reference to preferred embodiments thereof, it will be understood by those skilled in the art that the foregoing and other changes in form and details may be made therein without departing from the spirit and scope of the invention. For example, it should be clearly understood that the assembly of the present invention is not limited to use on chronic total occlusions. It may be used to treat acute total occlusions or other forms and/or degrees of artery blockage. It should also be clearly understood that the assembly of the present invention is not limited to use on peripheral arteries. It may be adapted for use on coronary arties or for veins.

What is claimed is:

1. An assembly for crossing hardened plaque comprising, in combination:
a centering catheter; and
a crosser having means for crossing said hardened plaque, said crosser dimensioned to pass through said centering catheter.

2. The assembly of claim 1 wherein said hardened plaque being a chronic total occlusion.

3. The assembly of claim 1 wherein said centering catheter having a centering balloon coupled about a shaft portion of said centering catheter and coupled proximate a distal end of said centering catheter.

4. The assembly of claim 1 wherein said centering catheter comprises a hemostatic valve coupled to a proximal end of said centering catheter.

5. The assembly of claim 1 wherein said centering catheter comprises means for removal of at least one of blood and debris from a site of said hardened plaque.

6. The assembly of claim 1 wherein said means for crossing said hardened plaque being substantially corkscrew-shaped.

7. The assembly of claim 1 wherein said means for crossing said hardened plaque being substantially auger-shaped.

8. The assembly of claim 1 wherein said means for crossing said hardened plaque having helicoidal threads.

9. The assembly of claim 1 wherein said means for crossing said hardened plaque being substantially concave and substantially oblong.

10. The assembly of claim 1 wherein said means for crossing said hardened plaque having at least one substantially pointed tip.

11. The assembly of claim 1 wherein said means for crossing said hardened plaque being dimensioned to be advanced to a site of said hardened plaque through an aperture defined by a distal end of said centering catheter.

12. The assembly of claim 1 wherein said crosser further comprises a handle to aid twisting of said crosser in order to advance said means for crossing said hardened plaque through an aperture defined by a distal end of said centering catheter and toward a site of said hardened plaque.

13. The assembly of claim 1 further comprising a guide wire, said guide wire being dimensioned to pass through said crosser.

14. The assembly of claim 13 wherein said crosser further comprises tightening means for securing said guide wire within said crosser, said tightening means being coupled to a proximal end of said crosser.

15. An assembly for crossing a chronic total occlusion comprising, in combination:
a centering catheter; and
a crosser, said crosser being dimensioned to be passed through said centering catheter and said crosser comprising:
a proximal end;
a shaft portion coupled to and extending from said proximal end of said crosser;
a hollow lumen defined by and extending along the length of said crosser; and
a corkscrew-shaped distal end coupled to and extending from said shaft portion of said crosser.

16. The assembly of claim 15 wherein said centering catheter comprises an aspiration port for removal of at least one of blood and debris from a site of said chronic total occlusion comprising.

17. The assembly of claim 15 wherein said crosser further comprises a handle to aid twisting of said crosser in order to advance said corkscrew-shaped distal end through an aperture defined by a distal end of said centering catheter and toward a site of said chronic total occlusion.

18. The assembly of claim 15 further comprising a guide wire being dimensioned to pass through said crosser.

19. The assembly of claim 18 wherein said crosser further comprises a torque device coupled to said proximal end of said crosser for securing said guide wire in place.

20. The assembly of claim 18 wherein said centering catheter having a centering balloon coupled about a shaft portion of said centering catheter and coupled proximate a distal end of said centering catheter.

21. A method for crossing a chronic total occlusion comprising the steps of:
providing an assembly having:
a centering catheter having a centering balloon coupled about a shaft portion of said centering catheter and coupled proximate a distal end of said centering catheter; and
a crosser having means for crossing said chronic total occlusion, said crosser dimensioned to pass through said centering catheter;
advancing a guide wire through an artery to a site proximate said chronic total occlusion;
advancing said centering catheter over said guide wire and through said artery to said site proximate said chronic total occlusion;
inserting said crosser into said centering catheter over said guide wire;
inflating said centering balloon of said centering catheter within said artery;
advancing said means for crossing said chronic total occlusion through an aperture defined by said distal end of said centering catheter; and
engaging said chronic total occlusion with said means for crossing said chronic total occlusion.

22. The method of claim 21 wherein said means for crossing said chronic total occlusion is advanced through said chronic total occlusion by twisting a handle coupled to a shaft portion of said crosser.

23. The method of claim 21 further comprising the steps of:
inserting a guide wire into said crosser; and
advancing said guide wire through said means for crossing said chronic total occlusion so that said guide wire is placed proximate said site of said chronic total occlusion;
advancing said guide wire through said chronic total occlusion;
deflating said centering balloon;
withdrawing said crosser from said site of said chronic total occlusion;
performing at least one of an angioplasty, plaque excision, and a stent.

24. The method of claim 23 wherein the step of advancing said guide wire through said chronic total occlusion is performed substantially contemporaneously with the step of advancing said means for crossing said chronic total occlusion through said chronic total occlusion.

25. The method of claim 23 further comprising the step of securing said guide wire within said crosser with tightening means.

26. The method of claim 21 further comprising the step of removing at least one of blood and debris from said site of said chronic total occlusion.

27. A method for crossing a chronic total occlusion comprising the steps of:
providing an assembly having:
a centering catheter having a centering balloon coupled about said shaft portion of said centering catheter and coupled proximate said distal end of said centering catheter; and
a crosser, said crosser being dimensioned to be passed through said centering catheter and said crosser comprising:
a proximal end;
a shaft portion coupled to and extending from said proximal end of said crosser;
a hollow lumen defined by and extending along the length of said crosser
a handle coupled to said shaft portion of said crosser; and
a corkscrew-shaped distal end coupled to and extending from said shaft portion of said crosser;
advancing a guide wire through an artery to a site proximate said chronic total occlusion;
advancing said centering catheter over said guide wire and through said artery to said site proximate said chronic total occlusion;
inserting said crosser into said centering catheter over said guide wire;
inflating said centering balloon of said centering catheter within said artery;
twisting said handle to advance said corkscrew-shaped distal end of said crosser through an aperture defined by a distal end of said centering catheter; and
engaging said chronic total occlusion with said corkscrew-shaped distal end of said crosser.
